(19) United States
(12) Patent Application Publication
(10) Pub. No.: US 2005/0064020 A1
(43) Pub. Date: Mar. 24, 2005

(54) Method for producing silver-containing antimicrobial fabric

(76) Inventors: Robert L. Schuette, Boiling Springs, SC (US); Jason Kreider, Boiling Springs, SC (US); Robert J. Goulet, Woodruff, SC (US); K. Mark Wiencek, Inman, SC (US); Raymond C. Sturm, Spartanburg, SC (US); T. Andrew Canada, Campobello, SC (US)

Correspondence Address: Terry T. Moyer, P. O. Box 1927, Spartanburg, SC, 29304, US

(21) Appl. No.: 10/950,278

(22) Filed: Sep. 24, 2004

Related U.S. Application Data
(63) Continuation-in-part of application No. 10/640,918, filed on Aug. 14, 2003.
(63) Continuation-in-part of application No. 10/640,919, filed on Aug. 14, 2003.
(63) Continuation-in-part of application No. 10/640,837, filed on Aug. 14, 2003.

(51) Int. Cl. A61K 33/38; A61K 33/42; A61K 9/70

(52) U.S. Cl. 424/443; 424/618; 424/604; 442/123

(57) ABSTRACT

Antimicrobial fabrics having a topically applied silver-based antimicrobial finish are provided. The finish comprises at least one silver ion-containing compound and at least one binder compound. The antimicrobial fabric may be formed into a garment to be worn as a base layer garment, close to the skin, which aids in the prevention of skin infection caused by abrasions to the skin. The garment may also aid in preventing the transfer of microbes from one person to another, for instance, after sharing communal items such as protective athletic equipment. The antimicrobial fabric exhibits long lasting antimicrobial efficacy against both Gram positive and Gram negative microbes and also exhibits antimicrobial efficacy after repeated wash cycles. Also provided is a method for making the silver-containing antimicrobial fabric.

### CROSS-REFERENCE TO RELATED APPLICATIONS

This application claims priority to and is a continuation-in-part of three co-pending U.S. patent applications having Ser. Nos. 10/640,918, 10/640,919, and 10/640,837, all of which were filed on Aug. 14, 2003.

### FIELD OF THE INVENTION

This invention relates to antimicrobial fabrics having a topically applied silver-based antimicrobial finish. The antimicrobial fabric exhibits long lasting antimicrobial efficacy against both Gram positive and Gram negative microbes and also exhibits antimicrobial efficacy after repeated wash cycles. Also provided is a method for making the silver-containing antimicrobial fabric.

In one potentially preferred embodiment, a silver-based antimicrobial finish is topically applied to a warp knit fabric comprised of polyester and spandex fibers. The treated fabric may ideally be made into a close-fitting base layer garment, such as an undershirt. Such a close-fitting garment enables the medicinal properties of the antimicrobial finish to easily contact the skin surface, thereby preventing or inhibiting skin infections caused by abrasion or transfer of microbes. For instance, the antimicrobial garment may be worn by military personnel to aid in the prevention of skin infection which often results from skin abrasions due to the continuous wearing of heavy equipment. Additionally, the garment may be ideal in assisting with the prevention of skin infection encountered in athletic sports such as football. In such circumstances, football players may be exposed to microbes, like Staphylococcus aureus, that already exist on the protective football equipment that is communally shared among the team members. Alternatively, the football players may develop skin abrasions from wearing the heavy protective equipment, and thus, are develop skin infections. It is contemplated herein that the base layer garment, especially if worn immediately next to the skin, will aid in preventing, or inhibiting, such skin infections. Additionally, such a fabric might have end-uses in the prevention of detection by reducing or eliminating odors, particularly human body odor. Such end-uses might include military special forces and hunting apparel.

### BACKGROUND OF THE INVENTION

Silver-containing microbicides have been incorporated into textile substrates for some time and are rapidly gaining acceptance in the medical industry as a safe, effective means of controlling microbial growth. It has long been recognized that silver plays an important role in promoting healing and in the prevention of infections. For example, U.S. Pat. No. 3,930,000 discloses the use of a silver zinc allantoinate cream for killing bacteria and fungi associated with burn wounds, and Japanese Abstract 09078430A discloses the incorporation of zirconium phosphate carrying silver into a thermoplastic olefin-based polymer melt for the extrusion of a synthetic antimicrobial fiber. Thus, it is known that placing surface available silver in contact with a wound allows the silver to enter the wound and become ingested by undesirable bacteria and fungi that grow and prosper in the warm, moist environment of the wound site. Once ingestion occurs, the silver kills the bacteria and fungi, which aids in preventing infection of the wound and promotes the healing process.

Much attention has been given recently to microbial skin infection outbreaks encountered by athletic sports players in many schools across the country. An article posted Oct. 31, 2003 on www.msnbc.msn.com/id/3226747 entitled, “Warning On Skin Infections in Athletes” acknowledges the increasing occurrence of skin infections among athletes, especially with regard to Staphylococcus aureus. It has been found that microbes are spread easily by athletes sharing equipment, using the same towel, or even sitting on the same bench. If not treated, or prevented at the onset, the skin infections can become much more serious and lead to infections of the blood, bones, or heart.

Additionally, since the antimicrobial fabric may be made into a garment, it may be important that the fabric exhibits antimicrobial efficacy after repeated wash cycles. In some instances, the garment may be a close fitting base layer worn by athletes under their protective gear which is worn for one day, washed, and then worn for another day. In other embodiments, the garment may be disposable and need not exhibit such wash durability characteristics. For example, military personnel engaged in conflict may wear the garment for several days and then discard it because of the inability to wash it and wear it again. Accordingly, the antimicrobial fabric should exhibit antimicrobial efficacy for an extended period of time.

With the potential for microbial growth at the site of a skin infection, another desirable feature of an antimicrobial fabric is that it absorbs odors emitted by the site. Especially since many of these skin infections occur on the upper body and are almost always covered by clothing, the lack of oxygen to the skin may lead to additional bacterial and/or fungal growth. This growth, quite often, leads to more severe infection of the skin abrasion and the creation of undesirable odors. Accordingly, it is desirable that the antimicrobial fabric possesses the capability of controlling odor due to the skin infection itself or due to other body malodors.

A topical treatment for textile substrates, such as a fabric, is desirable because it permits treatment of a fabric's individual fibers before or after weaving, knitting, and the like, in order to provide greater versatility to the target yarn without altering its physical characteristics. Such a coating, however, should prove to be successful at releasing a controlled amount of silver to a skin abrasion site while providing odor control and, for in some end-use applications, wash durability to be considered functionally acceptable. Furthermore, it is desirable for such a metallized treatment to be electrically non-conductive on the target fabric, fiber, or yarn surfaces. With the presence of metals and metal ions, it has been difficult in the past to obtain such a functional, electrically non-conductive coating for use in textile substrates.

Successful attempts at topically applying a silver-based antimicrobial finish to textile substrates are described in commonly assigned U.S. Pat. No. 6,584,668 to Green et al. and in commonly assigned U.S. patent application Ser. No. 09/586,381 to Green et al.; Ser. No. 09/586,081 to Green et al.; Ser. No. 09/589,179 to Green et al.; Ser. No. 09/585,762 to Van Hyning; Ser. No. 10/307,027 to Kreider et al.; Ser. No. 10/306,968 to Kreider et al.; Ser. No. 10/640,918 to Canada et al.; Ser. No. 10/640,919 to Canada et al.; and Ser. No. 10/640,837 to Canada et al. All of these patents and patent applications are herein incorporated by reference. The details of many of these processes will be discussed below.

Thus, the current invention discloses a method for achieving an antimicrobial fabric having a silver-based antimicrobial finish, which is topically applied to a target substrate. The resultant antimicrobial fabric provides controlled release of silver to the site of skin abrasion to aid in the prevention or treatment of skin infection and further provides protection against the transfer of microbes from one person to another. The antimicrobial fabric also exhibits odor control for eliminating or reducing undesirable odor emitted from the site of a skin infection and/or from other body malodors. While antimicrobial fabrics have been shown to inhibit odor, none have been produced which have been shown to solve the problems associated with preventing skin infection caused by skin abrasion and preventing transfer of microbes from one person to another through, for example, contaminated protective equipment.

### BRIEF DESCRIPTION OF THE DRAWINGS

FIG. 1 shows the results of zone of inhibition testing for inventive Example 1, when tested against Staphylococcus aureus ATCC #6538 on TSA with TCC plate.

FIG. 2 shows the results of zone of inhibition testing for Example 1, when tested against Staphylococcus aureus ATCC #6538 on DST agar plate.

FIG. 3 shows the results of zone of inhibition testing for Example 1, when tested against Klebsiella pneumoniae #4362 on TSA with TCC plate.

FIG. 4 shows the results of zone of inhibition testing for Example 1, when tested against Klebsiella pneumoniae #4362 on DST agar plate.

### DETAILED DESCRIPTION OF THE INVENTION

Substrate

Suitable substrates for receiving a topically applied silver-based antimicrobial finish include, without limitation, fibers, yarns, and fabrics. Fabrics may be formed from fibers such as synthetic fibers, natural fibers, or combinations thereof. Synthetic fibers include, for example, polyester, acrylic, polyamide, polyolefin, polyaramid, polyurethane, regenerated cellulose, and blends thereof. More specifically, polyester includes, for example, polyethylene terephthalate, polytriphenylene terephthalate, polybutylene terephthalate, polylactic acid, and combinations thereof. Polyamide includes, for example, nylon 6, nylon 6,6, and combinations thereof. Polyolefin includes, for example, polypropylene, polyethylene, and combinations thereof. Polyaramid includes, for example, poly-p-phenyleneteraphthalamid (i.e., Kevlar®), poly-m-phenyleneteraphthalamid (i.e., Nomex®), and combinations thereof. Natural fibers include, for example, wool, cotton, flax, and blends thereof.

The fabric may be formed from fibers or yarns of any size, including microdenier fibers and yarns (fibers or yarns having less than one denier per filament). The fibers or yarns may have deniers that range from less than about 1 denier per filament to about 2000 denier per filament or more preferably, from less than about 1 denier per filament to about 500 denier per filament, or even more preferably, from less than about 1 denier per filament to about 300 denier per filament.

Furthermore, the fabric may be partially or wholly comprised of multi-component or bi-component fibers or yarns which may be splittable along their length by chemical or mechanical action. The fabric may be comprised of fibers such as staple fiber, filament fiber, spun fiber, or combinations thereof.

The fabric may be of any variety, including but not limited to, woven fabric, knitted fabric, nonwoven fabric, or combinations thereof. The fabric may optionally be colored by a variety of dyeing techniques, such as high temperature jet dyeing with disperse dyes, thermosol dyeing, pad dyeing, transfer printing, screen printing, or any other technique that is common in the art for comparable, equivalent, traditional textile products. If yarns or fibers are treated by the process of the current invention, they may be dyed by suitable methods prior to fabric formation, such as, for instance, by package dyeing or solution dyeing, or after fabric formation as described above, or they may be left undyed. The textile substrate may be dyed or colored with any type of colorant, such as, for example, pigments, dyes, tints, and the like. Other additives may be present on and/or within the textile substrate, including antistatic agents, brightening compounds, nucleating agents, antioxidants, UV stabilizers, fillers, permanent press finishes, softeners, lubricants, curing accelerators, and the like.

In one embodiment of the invention, a warp knit fabric is used to form the antimicrobial garment. More specifically, a tricot warp knit fabric is used. To create a warp knit fabric, the yarns generally run in lengthwise in the fabric. The yarns are prepared as warps on beams with one or more yarns for each needle. A tricot warp knit fabric is a run-resistant type of warp knitting in which single or double sets of yarns are used. While a potentially preferred tricot warp knit fabric has been described, it is believed that any warp knit fabric that has been treated with the silver-based antimicrobial chemistry described herein would fall within the scope of the present disclosure, as well as any of the above-mentioned textile substrate materials.

The particular warp knit fabric described above provides many advantages over materials previously used for antimicrobial textile substrates. First, the fabric is surprisingly absorbent, despite its synthetic content. Second, because the fabric is synthetic, the antimicrobial garment is very durable and generates less lint than its natural counterpart, representing a reduced likelihood of complications with further infection in at a skin abrasion site caused by the lint and fiber from the antimicrobial garment itself. Third, the fabric's warp knit construction allows the fabric to stretch and conform to the shape of the body, thereby allowing the surface-available silver present on the surface of the garment to physically contact areas of skin abrasion and/or infection. Thus, the medicinal properties of the antimicrobial garment may be better utilized. In addition, the fabric is quite thin and lightweight, as compared with traditional woven cotton fabrics. The thinness of the present fabric facilitates its use as a base layer fabric which may be comfortably worn under, for example, military or athletic protective equipment. Accordingly, because the garment will not significantly contribute to increased bulk and thickness already encountered from the equipment, the garment provides more comfort and ease of use for the person wearing it. These advantages represent a useful advancement over the prior art.

Antimicrobial and Other Agents

The particular treatment used herein comprises at least one type of silver-ion containing compounds, or mixtures thereof of different types. The term “silver-ion containing compounds” encompasses compounds that are either ion-exchange resins, zeolites, or, possibly, substituted glass compounds that release the particular metal ion bonded thereto upon the presence of other anionic species. The preferred silver-ion containing compound for this invention is an antimicrobial silver sodium hydrogen zirconium phosphate available from Milliken & Company, under the tradename AlphaSan®. Other potentially preferred silver-containing antimicrobials in this invention, including silver zeolites, such as those available from Sinanen under the tradename Zeomic® AJ, silver exchanged on calcium phosphate available from Sangi under the tradename of Apiscider, and silver glass, such as those available from Ishizuka Glass under the tradename Ionopure®, may be utilized either in addition to, or as a substitute for, the preferred species. Other silver ion containing materials may also be used. Various combinations of these silver containing materials may be made if it is desired to “tune” the silver release rate over time.

Generally, such a metal compound is added in an amount from about 0.01% to about 60% by total weight of the particular treatment composition; more preferably, from about 0.05% to about 40%; and most preferably, from about 0.1% to about 30%. Preferably, the metal compound is present in an amount from about 0.01% to about 60% of the weight of the fabric (owf), preferably from about 0.05% to about 30% owf, more preferably from about 0.1% to about 10% owf, and most preferably from about 0.5% to about 5.0% owf.

The binder material provides highly beneficial durability of the antimicrobial compound for the target substrate. Preferably, this component is a polyurethane-based binding agent, although other binders, such as a permanent press type resin or an acrylic type resin, may also be used in combination, particularly with a halide ion additive for discoloration reduction. In essence, such resins provide durability by adhering silver to the target substrate, such as fibers or fabrics, with the polyurethane exhibiting the best overall performance.

The odor receiving agent can be a odor absorbing agent, and/or an odor adsorbing agent. Odor absorbing agents receive the odor and trap that odor inside the agent. Odor adsorbing agents receive the odor and hold the odor on the exterior of the agent. The odor receiving agent can be a particulate odor receiving agents, such as activated carbon, charcoal, zeolite compounds, or the like. Particulate odor receiving agents provide a greater surface area for receiving the odorous material. A carbonaceous material that can be converted into an activated carbon for the present invention include materials such as coal (bituminous), coconut shells, coke, peat, petroleum fractions, wood chips (saw dust), or the like. Other less common materials that can be used for forming activated carbon include automobile tires, cherry stones, coffee grounds, corn cobs, plastic waste, sewage sludge, straw, water lilies, or the like. Performance of the activated charcoal is typically improved with greater pore size and surface area. Generally, the smaller the particulate size, the better the odor receiving capability of the odor receiving agent.

Total add-on levels of silver to the target substrate may be 100 ppm or higher. More preferably, total add-on levels of silver may be 500 ppm or higher. It has not been determined that an upper boundary limit of silver add-on levels to the target substrate exist. However, consideration should be taken of the skin infection itself and prevention of any irritation to the site, or to the person wearing the antimicrobial garment, from excessive silver should be avoided.

Application Method

The preferred procedure utilizes silver-ion containing compounds, such as either AlphaSan®, Zeomic®), or Ionopure® as preferred compounds (although any similar types of compounds that provide silver ions may also be utilized), which are admixed with a binder to form a bath, into which the target substrate is then immersed.

It was initially determined that proper binder resins could be selected from the group consisting of nonionic permanent press binders (i.e., cross-linked adhesion promotion compounds, including, without limitation, cross-linked imidazolidinones available from Sequa under the tradename Permafresh®) or slightly anionic binders (including, without limitation, acrylics such as Rhoplex® TR3082 from Rohm & Haas). Other nonionics and slightly anionics were also suitable, including melamine formaldehyde, melamine urea, ethoxylated polyesters (such as Lubril QC™, available from Rhodia), and the like. However, it was found that the durability and controlled silver release of such treated substrates was limited.

It was determined that greater durability and control over silver release was required for this type of antimicrobial garment application. It is desirable that the antimicrobial fabric exhibits a controlled release of silver ions such that the silver ions are slowly released over an extended period of time, rather than being released quickly at one time. Thus, these prior comparative treatments were measured against various other types. Finally, it was discovered that certain polyurethane binders (such as Witcobond® from Crompton Corporation) and acrylic binders (such as Hystretch®) from BF Goodrich) permitted the best overall durability and controlled release of silver ion.

With such specific polyurethane-based binder materials utilized, the antimicrobial characteristics of the treated substrate remained very effective with regard to the amount of surface available silver that could be controllably released to kill bacteria, without discoloration of the treated substrate. However, while it currently appears that the use of polyurethane based binder resins are preferred due to their silver release and bio-neutral properties, in practice essentially any binder resin which is not toxic to the site of skin abrasion and/or infection may be used.

An acceptable method of providing a durable antimicrobial metal-treated fabric surface, is the application of a silver-ion containing compound and polyurethane-based binder resin from a bath mixture. In practice, this mixture of compound and resin may be applied through spraying, dipping, padding, foaming, and the like.

It has been recognized that silver-ion topical treatments are susceptible to yellowing, browning, graying, and, possibly, blacking after exposure to atmospheric conditions. As silver ions are generally highly reactive with free anions, and most anions that react with silver ions produce color, a manner of curtailing, if not outright preventing, problematic color generation upon silver ion interactions with free anionic species, particularly within dye bath liquids, was required. Thus, it was theorized that inclusion of an additive that was non-discoloring itself, would not react deleteriously with the binder and/or silver-ion compound, and would apparently, and without being bound to any specific scientific theory, react in such a manner as to provide a colorless salt with silver ions, was highly desired. It should be noted, however, that in some end-use applications, the prevention of discoloration may be less important, and the need for an additive which reduces discoloration may not be necessary.

Several methods for achieving this result are described in commonly assigned U.S. patent application Ser. Nos. 10/307,027; 10/306,968; and 10/418,019, all of which are entirely incorporated by reference herein. These Applications describe methods of including halide ions, such as from metal halides like magnesium chloride, in the silver-ion topical treatment to react with silver ions to produce colorless salts. Other examples include calcium chloride and ammonium chloride.

The inclusion of halide ions, such as from metal halides (for example, magnesium chloride) or hydrohalic acids (for example, hydrogen chloride) provide such results, with the exception that the presence of sodium ions (which are of the same valence as silver ions, and compete with silver ions for reaction with halide ions) should be avoided, since such components prevent the production of colorless silver halides, leaving the free silver ions the ability to react thereafter with undesirable anions. Thus, the presence of monovalent sodium ions (as well as other monovalent alkali metal ions, such as potassium, cesium, and lithium, at times) does not provide the requisite level of discoloration reduction. In general, amounts of 20 ppm or greater of sodium ions within the finish composition, particularly within the solvent (water, for example) are deleterious to the discoloration prevention of the topically applied antimicrobial treatments. Thus the term “substantially free from sodium ions” is used to indicate a presence of no more than this threshold amount of 20 ppm, and, more preferably, no more than 5 ppm.

Furthermore, the divalent or trivalent (and some monovalent) metal halide counteracts some effects of sodium ion exposure if present in a sufficient amount within the finish composition. Thus, higher amounts of sodium or like alkali metal ions are present within the finish composition; higher amounts of metal halide, such as magnesium chloride, for example, can counterbalance the composition to the extent that discoloration can be properly prevented. Additionally, all other metal ions—whether divalents, trivalents, and the like, with divalents, such as magnesium, being most preferred—combined with halide anions (such as chlorides, bromides, iodides, as examples, with chloride most preferred), as well as acids (such as HCl, HBr, and the like), are potential additives for discoloration prevention.

The concentrations of chloride ion should be measured in terms of molar ratios with the free silver ions available within the silver-ion containing compound. A range of ratios of chloride to silver ions should be from 1:10 to 5:1 for proper discoloration prevention; preferably, the range is from 1:2 to about 2.5:1. Again, higher amounts of metal halide in molar ratio to the silver ions may be added to counteract any excess alkali metal ion amounts within the finish composition itself.

The following Examples further illustrate the features of the present antimicrobial fabric but are not to be construed as limiting the invention as defined in the claims appended hereto. All parts and percents given in these examples are by weight unless otherwise indicated.

The fabric used in the Examples below was a tricot warp knit fabric, available from Milliken & Company of Spartanburg, S.C., having a fabric weight of about 8.6 ounces per linear yard. The fabric was comprised of continuous 40 denier/24 filament cationic dyeable polyester fiber and 40 denier spandex fiber. The polyester fiber comprised 79% of the warp knit fabric, while the spandex comprised 21% of the warp knit fabric. The fabric was jet dyed green using standard techniques and equipment known to those skilled in the art.

An antimicrobial finish containing AlphaSan® silver-based ion exchange compound (available from Milliken & Company of Spartanburg, S.C.) was produced for topical application to the target substrate. The formulation is as follows:

ANTIMICROBIAL FINISH FORMULATION

| Component | Amount (%) |
|---|---|
| Water | 82.9 |
| AlphaSan ® RC 2000 (10% Ag antimicrobial | 13.0 |
| agent) | |
| Witcobond ® 293 (polyurethane binder) | 5.0 |
| 30% Magnesium Chloride solution | 0.1 |

### EXAMPLE 1

The formulation was applied to the warp knit fabric via pad and nip rolls. The wet pickup on the fabric was approximately 30-35%. Example 1 was tested for a variety of characteristics as described below.

### EXAMPLE 2

The formulation was applied to the warp knit fabric via foam application to the face of the fabric. Example 2 was tested for a variety of characteristics as described below.

### EXAMPLE 3

The formulation was applied to the warp knit fabric via foam application to the back of the fabric. Example 3 was tested for a variety of characteristics as described below.

Cold Home Wash Procedure (AATCC Method 130-1995)

Example 1 was tested for wash durability with regard to antimicrobial efficacy against both Staphylococcus aureus and Klebsiella pneumoniae. The wash procedure was performed according to MTCC Method 130-1995 using water having a temperature of between about 65 and about 70 degrees F.

Test Microbes

Gram positive and Gram negative microbes were chosen to illustrate the effectiveness of the antimicrobial finish topically applied to the fabric to both types of organisms. Gram positive organisms include, for example and without limitation, Staphylococcus aureus, Clostridium perfringens, and Bacillus cereus. Gram negative organisms include, for example and without limitation, Klebsiella pneumoniae, Escherichia coli, and Pseudomonas aeruginosa. In the Examples illustrated below, Staphylococcus aureus and Klebsiella pneumoniae were selected for antimicrobial efficacy testing. However, it should be understood to be within the scope of this invention that other Gram positive and Gram negative organisms would exhibit antimicrobial efficacy results similar to those illustrated by the Examples below.

Zone of Inhibition Test

Example 1 was tested against Staphylococcus aureus ATCC #6538 and Klebsiella pneumoniae ATCC #4362 using a standard zone of inhibition test based on the Kirby-Bauer Agar-Diffusion Assay (Bauer A W, Kirby W M, Truck M. “Antibiotic susceptibility testing by a standardized single disc method.” American Journal of Clinical Pathology 1966; 45: 493.). Petri dishes containing Tryptic Soy Agar (TSA) or Diagnostic Sensitivity Test (DST) agar were inoculated via spreading with 0.5 ml of a diluted overnight culture of approximately 5E5 cells/ml into 100 mM Na/K phosphate buffer of the test organism. An approximately 1 inch by 1 inch piece of Example 1 fabric was then placed at the center of each agar plate. The agar plates were incubated for 24 hours at 37 degrees C. In some cases, an untreated fabric made of the same construction as in Example 1, but without the antimicrobial, also was tested.

Tryptic Soy Agar was supplemented with 0.01% Triphenyltetrazolium chloride (TTC). TTC is a colorless compound that is reduced to an insoluble red color by actively metabolizing bacteria. The plate was incubated for 24 hours and observed for TTC red colony formation. The zone of inhibition assay (“ZOI Assay”) provides both a qualitative (level of growth underneath sample) and quantitative (size of zone in mm) assessment of the performance of an antimicrobial agent incorporated into a fabric. The level of growth underneath the sample can be rated from confluent (no activity), to spotty or isolated (bacteriostatic), to nil (bactericidal). If reduced growth is observed underneath the sample for a particular microorganism compared to an untreated control dressing, that microorganism is considered sensitive and the antimicrobial agent is effective (bacteriostatic). The magnitude of the zone of inhibition, if one is observed, is a measure of both the inherent efficacy of the agent and the diffusion of the agent through the nutrient agar matrix. Generally, the larger the zone of inhibition, the more effective the fabric sample is at killing the bacteria. This zone of inhibition assay can be used to measure the efficacy of the antimicrobial fabric in a simulated clinical application by subjecting the fabric to multiple insults of a high level of bacteria over a period of seven days (indicated as “Exposure Event” in Tables 2A and 2B).

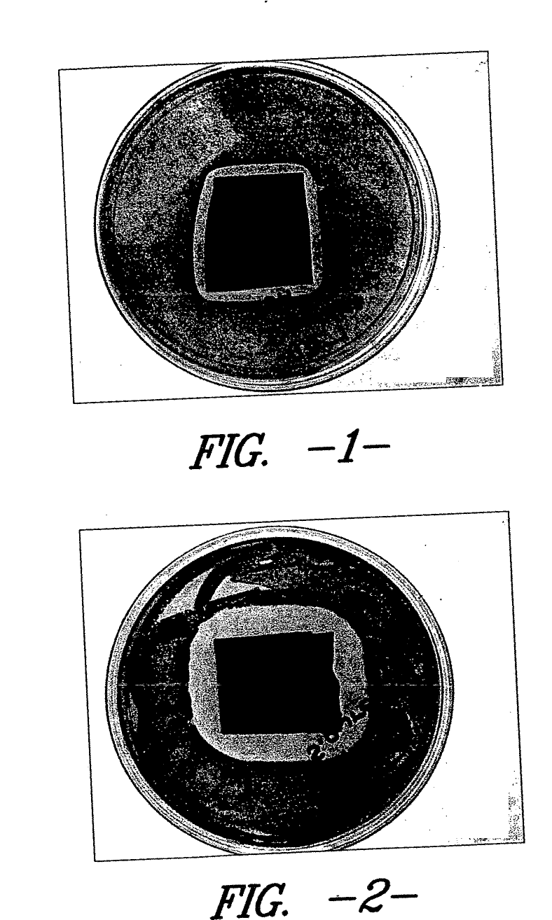

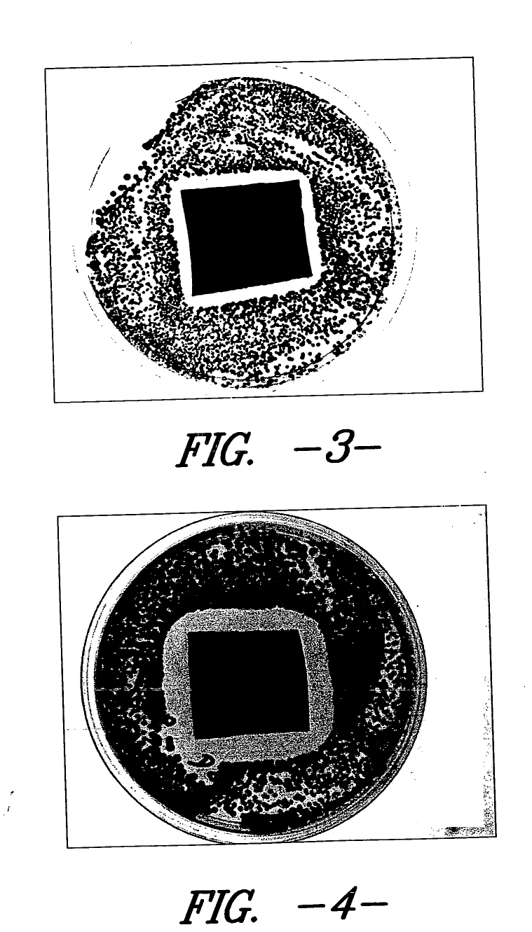

The results shown in Tables 1A and 1B below, represented by an average of 4 measurements (1 measurement from each of 4 sides of the square sample), and in FIGS. 1-4, demonstrate that inventive Examples 1-3, which contained AlphaSan® RC 2000, were antimicrobially active against both test microbes with the two different agar media. ZOls on TSA/TTC media generally were lower than with DST media. This result is believed to be caused by formulation differences in the media allowing silver ions to migrate to a greater distance on DST agar media. Both the face and the back side of the fabric exhibited considerable efficacy with ZOls in the 6-8 mm range. Slightly higher ZOls were measured on the face of the fabric when compared to the back of the fabric. In previous tests with untreated fabric, no ZOI or inhibition of growth underneath the sample was observed (data not shown).

TABLE 1A
Antimicrobial Efficacy Against Staphylococcus aureus
As Determined By Zone of Inhibition

| | | Average | | |
|---|---|---|---|---|
| | | Day 1 | Day 1 | |
| | Agar | Zone | Growth | Day 1 Swab |
| Sample | Plate | (mm) | Results | Conclusion |
| Example 1 | TSA/TTC | 3 | No | Bactericidal |
| | | | Growth | |
| Example 1 | DST | 8 | No | Bactericidal |
| | | | Growth | |
| Example 2 | DST | 7 | No | Bactericidal |
| | | | Growth | |
| Example 3 | DST | 6 | No | Bactericidal |
| | | | Growth | |

TABLE 1B
Antimicrobial Efficacy Against Klebsiella pneumoniae
As Determined By Zone of Inhibition

| | | Average | | |
|---|---|---|---|---|
| | | Day 1 | Day 1 | |
| | Agar | Zone | Growth | Day 1 Swab |
| Sample | Plate | (mm) | Results | Conclusion |
| Example 1 | TSA/TTC | 4 | No | Bactericidal |
| | | | Growth | |
| Example 1 | DST | 7 | No | Bactericidal |
| | | | Growth | |
| Example 2 | DST | 8 | No | Bactericidal |
| | | | Growth | |
| Example 3 | DST | 7 | No | Bactericidal |
| | | | Growth | |

Repeated Zone of Inhibition Test

Example 1 was tested against Staphylococcus aureus ATCC #6538 and Klebsiella pneumoniae ATCC #4362 using a standard zone of inhibition test based the Kirby-Bauer Agar-Diffusion Assay. An overnight culture of the test microbe was diluted into 100 mM Na/K phosphate buffer to a concentration of approximately 5E6 cells/ml. Petri dishes containing Diagnostic Sensitivity Test (DST) agar were inoculated with 0.1 ml of the cell suspension and incubated for 1 hour. An approximately 1 inch by 1 inch piece of Example 1 fabric was then placed at the center of each agar plate. The agar plate was incubated for 24 hours at 37 degrees C. After measuring the zone, the fabric was transferred to a fresh DST plate and inoculated with the same microbe as described above. The fabric was exposed to fresh agar plates seven times over a period of ten days. Accordingly, the zone of inhibition assay can be used to measure the efficacy of the antimicrobial fabrics in a simulated clinical application by subjecting the fabric to multiple insults of a high level of bacteria over an extended period of time. Generally, the larger the zone of inhibition, the more effective the fabric sample is at inhibiting the growth of the bacteria.

The results shown in Tables 2A and 2B below, represented by an average of 4 measurements from 4 sides of the square sample, demonstrate that inventive Example 1, which contained AlphaSan® RC 2000, was antimicrobially active against the various types of bacteria in repeated exposures after home washing. The unwashed fabric exhibited antimicrobial efficacy through 5 exposures, with ZOls decreasing over time. Efficacy of washed samples was good for 1 exposure, but disappeared relatively quickly with subsequent exposures. In previous tests with untreated fabric, no ZOI or inhibition of growth underneath the sample was observed (data not shown).

The wash durability of Example 1 was illustrated by a zone of inhibition for both Gram positive and Gram negative microbes of at least 1 millimeter after at least 1 home wash cycle. However, Tables 2A and 2B show that Example 1 exceeded this minimum requirement and remained wash durable against both Gram positive and Gram negative microbes with ZOls of at least 5 millimeters after 5 home wash cycles. Test data indicated as “nd” means “not determined.”

While the results In Tables 2A and 2B below illustrate that the antimicrobial finish is wash durable, which may be important in some end-use applications, it is also contemplated that a disposable antimicrobial garment may be desirable. In such cases, wash durability properties may not be as important. Instead, it may be most desirable that the garment exhibits controlled release of silver over an extended period of time and with repeated exposure to bacteria, as shown by Example 1 in Tables 2A and 2B after no home wash cycles.

TABLE 2A
Wash Durability of Antimicrobial Efficacy Against Staphylococcus aureus
As Determined By Zone of Inhibition

| | # Cold | Exposure | Exposure | Exposure | Exposure | Exposure | Exposure | Exposure |
|---|---|---|---|---|---|---|---|---|
| | Home | Event 1 | Event 2 | Event 3 | Event 4 | Event 5 | Event 6 | Event 7 |
| Sample | Washes | Zone (mm) | Zone (mm) | Zone (mm) | Zone (mm) | Zone (mm) | Zone (mm) | Zone (mm) |
| Example 1 | 0 | 10 | 7 | 6 | 5 | 3 | 0 | 0 |
| Example 1 | 1 | 7 | 2 | 0 | 0 | 0 | 0 | 0 |
| Example 1 | 3 | 6 | 0 | 0 | nd | nd | nd | nd |
| Example 1 | 5 | 5 | 0 | 0 | 0 | nd | nd | nd |

TABLE 2B
Wash Durability of Antimicrobial Efficacy Against Klebsiella pneumoniae
As Determined By Zone of Inhibition

| | # Cold | Exposure | Exposure | Exposure | Exposure | Exposure | Exposure | Exposure |
|---|---|---|---|---|---|---|---|---|
| | Home | Event 1 | Event 2 | Event 3 | Event 4 | Event 5 | Event 6 | Event 7 |
| Sample | Washes | Zone (mm) | Zone (mm) | Zone (mm) | Zone (mm) | Zone (mm) | Zone (mm) | Zone (mm) |
| Example 1 | 0 | 10 | 8 | 6 | 7 | 5 | 2 | 0 |
| Example 1 | 1 | 8 | 3 | 0 | 0 | 0 | 0 | 0 |
| Example 1 | 3 | 6 | 0 | 0 | nd | nd | nd | nd |
| Example 1 | 5 | 6 | 0 | 0 | nd | nd | nd | nd |

Silver Elution Test

Example 1 was tested to determine its ability to controllably release surface available silver.

A 10× strength stock extraction solution of a phosphate buffer solution (PBS) was prepared by combining (in a 1L flask) 144.46 g of sodium phosphate with 71.18 g of potassium phosphate. Deionized water was then added to the 1 L flask until the flask contained a total volume of 1000 ml. The contents of the flask were mixed with a stir bar until all salts were completely dissolved. The 10× PBS stock extraction solution was then diluted to 1× by diluting 100 ml of PBS 10× stock to 1000 ml using deionized water.

Ten grams of the fabric was then immersed in a container holding 100 mL of the 1×PBS extraction buffer for 24 hours at 37 degrees C. The extraction solution was then analyzed by Atomic Absorption Spectrophotometer for a measurement of available silver removed from the surface of the fabric.

Example 1 controllably released 7.3 μg of silver per square centimeter of fabric from its surface in a 24 hour period. Accordingly, it may be desirable that the antimicrobial fabric release less than about 50 μg/cm² of silver over a 24 hour period. It may be more preferable that the antimicrobial fabric release less than about 25 μg/cm² of silver over a 24 hour period.

Furthermore, it may be most preferable that the antimicrobial fabric release less than about 10 μg/cm² of silver over a 24 hour period.

Total ALPHASAN® Content Test

The amount of active ALPHASAN® compound transferred to the fabric of Example 1 in the application process was determined using the following Ash Procedure technique.

In the Ash Procedure technique, a sample of fabric (weighing approximately 10 grams and measured to four significant digits) was placed in a clean, dry crucible. The crucible containing the fabric sample was placed in a muffle furnace whose temperature ramped up at 3° C./minute to 750° C. The temperature was then held at 750° C. for one hour. The system was then cooled and the crucible transferred to a desiccator in which it was allowed to reach an equilibrium temperature. The crucible was then weighed.

In the Ash Digestion technique, the fabric sample was then ground in the crucible to obtain a uniform sample of approximately 0.1 g weight (again measured to four significant digits). Four milliliters of 50% HNO₃, followed by 10 drops of 48% HF, were added to the sample. The sample was heated over a hot plate in a platinum crucible until it completely dissolved. The sample solution was then transferred to a 100 mL volumetric flask.

The crucible was then rinsed with 5% HNO₃, with the rinse solution being added to the flask.

The solution was diluted to the 100 mL mark with 5% HNO₃. The dilute solution was transferred to a polyethylene storage container. Analysis for the desired active ingredient (in this case, silver) was performed using an Inductively Coupled Plasma device (e.g., a Perkin Elmer Optima 4300 DV). Calculations are apparent to one skilled in the art.

Example 1 exhibited 1.57% active ALPHASAN® compound (i.e. total silver) on weight of the fabric.

The test data demonstrates the inventive silver-containing antimicrobial fabric having a topically applied antimicrobial finish effectively inhibits the growth of both Gram positive and Gram negative bacteria (a) over repetitive exposure events and (b) after repeated wash cycles. Additionally, the fabric exhibits controlled release of silver, since no immediate dumping of excessive amounts of silver occurred during the 24 hour silver elution test. Thus, the above description and examples show that a topical antimicrobial finish may be applied to a textile substrate to achieve an antimicrobially effective, wash durable, silver-containing garment having the desired characteristics of antimicrobial efficacy, controlled release of silver, odor absorption, and lack of discoloration.

Further, it is contemplated to be within the scope of the current invention that the antimicrobial finish may be tailored in order to obtain optimum performance for a particular end-use application. For example, a fabric's ability to wick moisture may be increased in order to cause a higher initial release of silver from the fabric, since moisture tends to draw out the release of silver from the surface of the fabric. This may be ideal for short-term use of a fabric, and possibly for disposable fabrics. Another option includes increasing the amount of magnesium chloride in the antimicrobial finish. This may lead to a decrease in silver release from the surface of the fabric. This may be ideal for long-term end-use applications and those applications where color stability is important. Thus, the presence and the exact amounts of the various components comprising the antimicrobial finish may be varied as necessary in order to obtain a silver-containing antimicrobial fabric that performs optimally for a specific end-use application.

These and other modifications and variations to the present invention may be practiced by those of ordinary skill in the art, without departing from the spirit and scope of the present invention. Furthermore, those of ordinary skill in the art will appreciate that the foregoing description is by way of example only, and is not intended to limit the scope of the invention described in the appended claims.

1. A process for producing a wash durable, silver-containing antimicrobial fabric having a surface, at least a portion of which is coated with a finish,
wherein said finish comprises at least one compound delivering silver ions and at least one binder material, and wherein said finish exhibits antimicrobial properties; and
wherein said coated antimicrobial fabric exhibits a controlled silver-ion release rate of less than about 50 μg/cm² of silver ions over a 24 hour period;
said process comprising the steps of:
(a) providing a fabric substrate;
(b) providing said finish, wherein said finish also comprises at least one binder material; and
(c) applying said finish to at least a portion of said fabric substrate without causing discoloration of said fabric substrate due to chemical instability of said finish.

2. The process of claim 1, wherein said coated antimicrobial fabric exhibits a controlled silver-ion release rate of less than about 25 μg/cm² of silver ion over a 24 hour period.

3. The process of claim 1, wherein said coated antimicrobial fabric exhibits a controlled silver-ion release rate of less than about 10 μg/cm² of silver ion over a 24 hour period.

4. The process of claim 1, wherein said finish is non-electrically conductive.

5. The process of claim 1, wherein said compound delivering silver ions is selected from the group consisting of silver zirconium phosphate, silver calcium phosphate, silver zeolite, silver glass, and any mixtures thereof.

6. The process of claim 5, wherein said compound delivering silver ions is silver zirconium phosphate.

7. The process of claim 1, wherein said at least one binder material is selected from the group consisting of polyurethane-based binders, acrylic-based binders, and permanent press-based binders.

8. The process of claim 7, wherein said at least one binder material is a polyurethane-based binder.

9. The process of claim 1, wherein said coated antimicrobial fabric exhibits a zone of inhibition against Gram positive microbes of between about 1 mm and about 10 mm.

10. The process of claim 1, wherein said coated antimicrobial fabric exhibits a zone of inhibition against Gram positive microbes of between about 1 mm and about 7 mm after 1 home wash cycle.

11. The process of claim 1, wherein said coated antimicrobial fabric exhibits a zone of inhibition against Gram positive microbes of between about 1 mm and about 6 mm after 3 home wash cycles.

12. The process of claim 1, wherein said coated antimicrobial fabric exhibits a zone of inhibition against Gram positive microbes of between about 1 mm and about 5 mm after 5 home wash cycles.

13. The process of claim 1, wherein said coated antimicrobial fabric exhibits a zone of inhibition against Gram negative microbes of between about 1 mm and about 10 mm.

14. The process of claim 1, wherein said coated antimicrobial fabric exhibits a zone of inhibition against Gram negative microbes of between about 1 mm and about 8 mm after 1 home wash cycle.

15. The process of claim 1, wherein said coated antimicrobial fabric exhibits a zone of inhibition against Gram negative microbes of between about 1 mm and about 6 mm after 3 home wash cycles.

16. The process of claim 1, wherein said coated antimicrobial fabric exhibits a zone of inhibition against Gram negative microbes of between about 1 mm and about 6 mm after 5 home wash cycles.

17. A process for producing a wash durable, silver-containing, warp knit antimicrobial fabric having a surface, at least a portion of which is coated with a finish,
wherein said finish comprises at least one compound delivering silver ions and at least one binder material, and wherein said finish exhibits antimicrobial properties; and
wherein said coated antimicrobial fabric exhibits a controlled silver-ion release rate of less than about 50 μg/cm² of silver ions over a 24 hour period;
said process comprising the steps of:
(a) providing a warp knit fabric;
(b) providing said finish, wherein said finish also comprises at least one binder material; and
(c) applying said finish to at least a portion of said warp knit fabric without causing discoloration of said warp knit fabric due to chemical instability of said finish.

18. A process for producing a silver-containing, warp knit antimicrobial fabric having a surface, at least a portion of which is coated with a finish,
wherein said finish comprises at least one compound delivering silver ions and at least one binder material, and wherein said finish exhibits antimicrobial properties; and
wherein said coated antimicrobial fabric exhibits a controlled silver-ion release rate of less than about 50 μg/cm² of silver ions over a 24 hour period, and
wherein said coated antimicrobial fabric exhibits a zone of inhibition for Gram positive and Gram negative microbes of at least 1 mm after 1 home wash cycle;
said process comprising the steps of:
(a) providing a warp knit fabric;
(b) providing said finish, wherein said finish also comprises at least one binder material; and
(c) applying said finish to at least a portion of said warp knit fabric without causing discoloration of said warp knit fabric due to chemical instability of said finish.

19. The product of the process of claim 1.

20. The product of the process of claim 17.

21. The product of the process of claim 18.
